(19) United States
(12) Patent Application Publication
(10) Pub. No.: US 2022/0249201 A1
(43) Pub. Date: Aug. 11, 2022

(54) DASHBOARD FOR VISUALIZING ORTHODONTIC METRICS DURING SETUP DESIGN

(71) Applicant: 3M INNOVATIVE PROPERTIES COMPANY, St. Paul, MN (US)

(72) Inventors: Vera Shuman, St. Paul, MN (US); Alexandra R. Cunliffe, St. Paul, MN (US); Guruprasad Somasundaram, Woodbury, MN (US); Benjamin D. Zimmer, Hudson, WI (US); Nitsan Ben-Gal Nguyen, Apple Valley, MN (US)

(21) Appl. No.: 17/263,622

(22) PCT Filed: Jul. 24, 2019
(86) PCT No.: PCT/IB2019/056342

§ 371 Date: Jan. 27, 2021

Related U.S. Application Data
(60) Provisional application No. 62/712,380, filed on Jul. 31, 2018.

(51) Int. Cl. A61C 7/00; G16H 20/40

(52) U.S. Cl. CPC A61C 7/002; G16H 20/40

(57) ABSTRACT

A method for evaluating intermediate and final setups for orthodontic treatment. The method includes receiving intermediate and final setups, where each setup is a digital representation of a state of teeth at a particular stage of orthodontic treatment. Scores are computed based upon metrics related to at least some of the states represented by the corresponding setups, and the scores provide an indication of a validity of the corresponding states. The metrics along with an indication of the corresponding scores are displayed in a dashboard in order to provide a visual evaluation of the validity of the setups.

### BACKGROUND

The goal of the orthodontic treatment planning process is to determine where the post-treatment positions of a person's teeth (setup state) should be, given the pre-treatment positions of the teeth in a malocclusion state. This process is typically performed manually using interactive software and is a very time-consuming process. A need thus exists for a final setup algorithm to serve as an efficiency enhancer, greatly reducing the time needed to process a case, along with a way to rate and evaluate treatment options.

### SUMMARY

A computer-implemented method for evaluating setups for orthodontic treatment of an embodiment of the invention includes receiving one or more setups, where each setup is a digital representation of a state of teeth at a particular stage of orthodontic treatment. Scores are computed based upon metrics related to at least some of the states represented by the corresponding setups, where the scores provide an indication of a validity of the corresponding states. The metrics along with an indication of the corresponding scores are displayed in order to provide a visual evaluation of the corresponding setups.

### BRIEF DESCRIPTION OF THE DRAWINGS

The accompanying drawings are incorporated in and constitute a part of this specification and, together with the description, explain the advantages and principles of the invention. In the drawings,

FIG. 1 is a diagram of a system for visualizing orthodontic metrics;

FIG. 2 is a diagram of another embodiment of the system of FIG. 1;

FIG. 3 is an example of a user interface displaying a heat map for orthodontic metrics;

FIG. 4 is an example of a user interface displaying a line graph showing treatment development at each intermediate stage;

FIG. 5 is an example of a user interface displaying a bar graph tracking current setup;

FIG. 6 is an example of a user interface displaying a bar graph comparing two treatment plans;

FIG. 7 is an example of a user interface displaying information about bite comfort based upon between-arch collisions; and

FIG. 8 is an example of a user interface displaying information about bite comfort based upon between-arch collisions and tooth movement speed.

### DETAILED DESCRIPTION

### Overview

Described herein is an information management system, methods, and visualization tools to track, analyze, and visualize key orthodontic performance indicators for orthodontic treatment planning that can be used to evaluate and adapt intermediate and final setups and to communicate this information effectively to a clinician and patient.

The dashboard would provide real-time feedback to a doctor, technician, or patient as to the quality of their orthodontic treatment plan, allowing the user to design treatments more quickly and compare treatment alternatives. It would also inform a user when a treatment plan is sufficiently correct, meaning that time need not be spent attempting to improve the case further.

Applications of this technology include a fully automated treatment planning workflow, an interactive workflow to aid a clinician during treatment planning, and/or a visual communication tool during doctor-patient or technician-doctor interaction.

Embodiments of this invention include a suite of scoring methods and visualization tools for orthodontic treatment planning. The scoring methods enable automated intermediate and final setup generation for clear tray aligners. However, they provide many other opportunities in both automated and interactive treatment planning, including: computation of quantitative metrics to assess treatment plans, visualization of treatment options and trade-offs, and assessment of long-term effects of treatment options on holistic oral health (e.g., occlusal disease).

Further described herein is a method to visualize these metrics during orthodontic treatment planning to aid a user in treatment design.

### Functions of the System Components

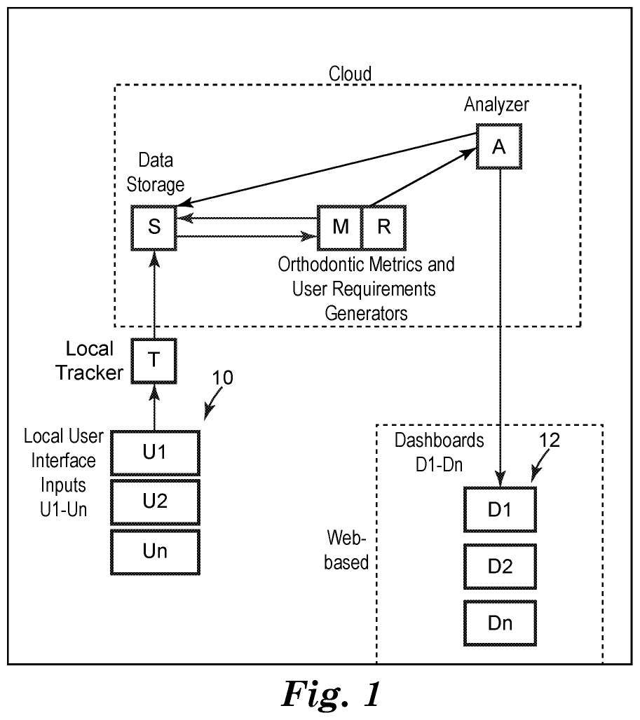

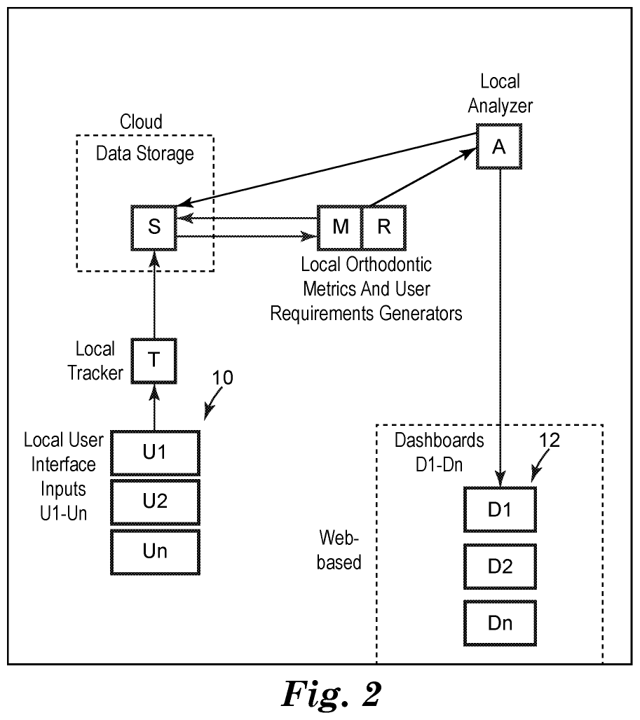

FIG. 1 is a diagram of a system for visualizing orthodontic metrics, and FIG. 2 is a diagram of another embodiment of the system of FIG. 1.
User Interface Inputs (U1-Un) receive from input devices 10 such as an interactive user interface displayed on an electronic display device.
   To upload tooth scans.
   To upload photos of the patient.
   To input tooth positions, landmarks, etc.
   To input prescription data.
   To input treatment time data.
   To input treatment cost data.
   To input other patient data.
   To input general user preferences (e.g., real time or on demand analysis)
   Sends data to one local Tracker (T).
   Local at each user station.
   Can be local or cloud based.
Local Trackers (T)
   To receive data from one User Interface Input (U).
   To store most current data.
   To send this data to the Data Storage (S).
   Local at each user station.
   Can be local or cloud based.
Data Storage (S)
   To receive data from local Trackers (Ts), the Orthodontic Metrics and User Requirements Generators (M, R), and the Analyzer (A).
   To store
   Final setups and intermediate setups data from patients.
   Treatment time data.
   Treatment cost data.
   Treatment metric data.
   Treatment requirements data.
   Evaluation results from the Analyzer.
   Whereby ‘patients’ includes other patients and current case.
   Whereby storage includes version controls of stored data.
   To send data to the Orthodontic Metrics Generator (M) and the User Requirements Generator (R).
   Cloud based.
Orthodontic Metrics Generator (M)
   To receive data from the Data Storage (S).
   To generate orthodontic metrics based on this data.
   To send these metrics to the Analyzer (A).
   To send these metrics to the Data Storage (S).
   Can be local or cloud based.
User Requirements Generator (R)
   To receive data from the Data Storage (S).
   To receive structured and unstructured prescription data, treatment time data, cost data, and user preference data.
   To generate user requirements based on these inputs.
   To send identified user requirements to the Analyzer (A).
   To send the identified user requirements to the Data storage (S).
   Can be local or cloud based.
Analyzer (A)
   To receive metrics data from the Orthodontic Metrics Generator (M) and the User Requirements Generator (R).
   To run evaluations functions based on current data, comparison data, and user requirements.
   To compute comparisons between different final setups and intermediate setups.
   To generate descriptive and inferential statistics.
   To compute anticipated treatment time.
   To compute anticipated cost.
   To send results to the Dashboard (D).
   To send results to the Data Storage (S).
   Can be local or cloud based.
Dashboards (D1-Dn) for display on display devices 12.
   To display final setups and intermediate setups.
   To display indicators of goodness of final setups and intermediate setups.
   E.g., ranking, pass/fail, heat map, trends, alerts etc. (see detailed description in text).
   To update data in real time or upon user input.
   Web-based.
   The dashboards can also supplement with the displayed information with auditory information provided, for example, via electronic speakers associated with the display devices 12.

The Orthodontic Metrics Generator (M), Analyzer (A), and User Requirements Generator (R) can be implemented with one or more processors or computing devices. The Local Trackers (T) and Data Storage (S) can be implemented with one or more electronic storage devices possibly with associated computing devices. The methods described herein can be implemented in software or firmware modules, for example, for execution by the processors or computing devices, and can alternatively be implemented in hardware modules or a combination of software and hardware.

### Orthodontic Metrics Generator

Scoring Based on Geometric Information

A number of scoring methods may be used describe the validity of a state (arrangement of teeth) based on collisions between teeth. For example, a larger score may indicate a state that is worse, while a smaller score indicates a state that is closer to valid, with zero being a valid state, or alternatively larger scores may indicate better states.
Collision count: the count of collisions (unique colliding pairs of teeth) in the state.
Penetration depth: the sum of penetration depths of all collisions in the state.
Collision contact points count: the total count of contact points in all collisions in the state. Collision contact points count may serve as an estimate of penetration since it will tend to increase as collisions get deeper.
Weighted sum of several of the metrics described above, e.g., α×Collision count+β×Penetration depth.

Scoring Based on Physical Information

The geometric information discussed above can be used to provide physical information to inform treatment planning and facilitate effective communication with clinicians and patients. Scores may also be combined with other information, including landmarks, tooth movement between states, and tooth position to provide holistic oral health and comfort information. Such a system would go beyond being an orthodontic tool and rather serve as a unified treatment platform for dentists, orthodontists, and others.

Examples of Specific Scores

A list and short description of various metrics that may be computed and displayed for intermediate and final setups are described in co-owned US patent application filed on even date here and entitled “Method for Automated Generation of Orthodontic Treatment Final Setups” (U.S. Provisional Application No. 62/712,383, filed Jul. 31, 2018), which is incorporated herein by reference as if fully set forth. These metrics may be presented individually, or they can be combined to report more abstract qualities about the treatment. For example:
Bite Comfort: A bite comfort metric may be computed based on the number of collisions between two arches, the penetration depth of each collision, and the location of each collision (e.g., rear molar versus incisor).
Bite Alignment: Inter-arch collisions may be combined with other metrics that indicate alignment between teeth on opposing arches, including class relationship, crossbite, overbite, and overjet. This single metric could be used to assess and report bite alignment.
Treatment comfort: A treatment comfort score may be computed based on the combination of two metrics:
   1. The bite comfort score at intermediate stages throughout treatment
   2. The ratio of tooth speed to the maximum allowable tooth speed when moving from one stage to another.
Orthodontic correctness: Metrics described herein can be combined to yield one or more scores to assess the orthodontic correctness of a single arrangement of teeth.

Bite comfort, bite alignment, treatment comfort, orthodontic correctness, and other metrics can also be used in a scoring function for final setup prediction.

### Dashboard

Orthodontic metrics can be presented to a user in the form of a dashboard displayed on an electronic display device within a screen or user interface. The dashboard should be designed to strike a balance between the complexity of orthodontic metrics on the one hand, and user needs and capabilities for information processing on the other hand. The visual information in the displayed dashboard can be supplemented by auditory information, for example the metrics and related information provided in audible form via an electronic speaker.

Different user groups could electronically access the dashboard, including technicians, doctors, and patients.

For technician and doctor use, this tool could be used to modify or aid in the design of the final treatment setup. For patient use, it could be used as a communication tool for the doctor to convince the patient to undergo treatment, and to set realistic expectations for treatment duration and anticipated success at various stages of the treatment. It could also be used to compare different courses of treatment, for example it could be used to highlight how a one year course of clear tray aligners could bring all visible metrics to green or yellow status, while a three month course of treatment may address some qualities and probably not all, for example midline may be addressed but not occlusal relationship. It could also be used to compare treatments with different modalities, for example clear tray aligners versus brackets and wires, and how each affects the results the patient will see and feel on a regular basis.

Given different user needs of these user groups, and the number and complexity of the metrics to be potentially displayed, the dashboard should be adaptable to different user requirements as follows:

| | Technician | Doctor | Patient |
|---|---|---|---|
| Primary user | Real time tracking | Evaluate final | Understand |
| need | of treatment | setup | treatment |
| | modifications | | plan |
| Secondary | Evaluate final setup | Compare | Compare |
| user needs | Compare | malocclusion with | treatment |
| | malocclusion with | final setup | alternatives |
| | final setup | Compare treatment | |
| | Compare treatment | alternatives | |
| | alternatives | Real time tracking | |
| | | of treatment | |
| | | modifications | |
| Complexity of | High complexity | High complexity | Low |
| dashboard | | | complexity |
| Integrated | yes | yes | no |
| with treatment | | | |
| design tool | | | |
| Adaptable to | yes | yes | no |
| individual | | | |
| user/case | | | |

To achieve a user friendly design, the underlying metrics can be simplified for visualization to the user. This can be achieved in a number of ways:

### Alerts

Alerts would be useful for technicians and doctors during treatment design and for treatment evaluations.

The dashboard can display only those metrics that show insufficient performance. It alerts the user when any of the metrics are much different than an ideal case. For instance, if contact between any two teeth exceeds 0.15 mm, the user could receive an alert regarding this metric. To reduce information overload, other metrics could remain hidden, or summarized in an “all other good” indicator.

The alert could be displayed in a separate designed field (e.g., upper right corner of the screen or user interface). Additionally, for technicians and doctors, a related action panel could be highlighted.

Alerts can be arranged sequentially by deviation value in the display panel to indicate which metrics are most different from the ideal. This would indicate a severity of the deviation, and therefore an order of importance in which to revisit and correct metrics.

### Heat Map

Colored fields ranging from green (all good) to yellow (caution) to red (alert) can be used to show to users in an intuitive view the status and change of multiple metrics when tracking treatment modifications, comparing malocclusions with final setups, comparing alternative treatment plans, and displaying anticipated treatment success over time to patients. The colored fields are represented herein by varying gray scale shades, and the status can be indicated by other colors or shading.

### Example: Tracking Treatment Modifications by a Technician or Doctor

| | Mal- | |
|---|---|---|
| | occlusion | Current |
| Alignment | red | green |
| Edge | red | green |
| alignment | | |
| Buccolingual | red | yellow |
| inclination | | |
| Class | green | green |
| relationship | | |
| Leveling | green | green |
| Mesial/distal | green | green |
| tilt | | |
| Relative | green | green |
| midline | | |
| Absolute | red | green |
| midline | | |
| Occlusal | red | yellow |
| contacts | | |
| Overbite | yellow | green |
| Overjet | green | green |
| Canine | green | green |
| overbite | | |
| Spacing | green | green |
| Collision | green | green |
| Contact | green | green |
| Bite | green | green |
| symmetry | | |
| Arch form | green | green |
| shape | | |
| Arch form | green | green |
| symmetry | | |

### Example: Treatment Comparison

| | Mal- | Treatment A | Treatment B |
|---|---|---|---|
| | occlusion | (saved) | (saved) |
| Alignment | red | yellow | green |
| Edge | red | green | green |
| alignment | | | |
| Buccolingual | red | green | yellow |
| inclination | | | |
| Class | green | green | green |
| relationship | | | |
| Leveling | green | green | green |
| Mesial/distal | green | green | green |
| tilt | | | |
| Relative | green | green | green |
| midline | | | |
| Absolute | red | green | green |
| midline | | | |
| Occlusal | red | yellow | yellow |
| contacts | | | |
| Overbite | yellow | yellow | green |
| Overjet | green | green | green |
| Canine | green | green | green |
| overbite | | | |
| Spacing | green | yellow | green |
| Collision | green | green | yellow |
| Contact | green | green | green |
| Bite symmetry | green | green | green |
| Arch form | green | green | green |
| shape | | | |
| Arch form | green | green | green |
| symmetry | | | |

### Example: Display Treatment Planning Over Time/Stages to Patient

| | Mal- | | | | | |
|---|---|---|---|---|---|---|
| | occlusion | Stage 1 | Stage 2 | Stage 3 | Stage 4 | Stage 5 |
| Alignment | red | red | yellow | yellow | green | green |
| Edge | red | red | red | yellow | green | green |
| alignment | | | | | | |
| Buccolingual | red | yellow | yellow | yellow | green | green |
| inclination | | | | | | |
| Class | green | green | green | green | green | green |
| relationship | | | | | | |
| Leveling | green | green | green | green | green | green |
| Mesial/distal | green | green | green | green | green | green |
| tilt | | | | | | |
| Relative | green | green | green | green | green | green |
| midline | | | | | | |
| Absolute | red | red | red | yellow | green | green |
| midline | | | | | | |
| Occlusal | red | red | red | yellow | yellow | green |
| contacts | | | | | | |
| Overbite | yellow | yellow | yellow | yellow | yellow | green |
| Overjet | green | green | green | green | green | green |
| Canine | green | green | green | green | green | green |
| overbite | | | | | | |
| Spacing | green | green | green | green | green | green |
| Collision | green | green | green | green | green | green |
| Contact | green | green | green | green | green | green |
| Bite | green | green | green | green | green | green |
| symmetry | | | | | | |
| Arch form | green | green | green | green | green | green |
| shape | | | | | | |
| Arch form | green | green | green | green | green | green |
| symmetry | | | | | | |

### Example: Display Treatment Planning Over Time/Stages to Patient with Tooth Information

| | Mal- | | | | | |
|---|---|---|---|---|---|---|
| | occlusion | Stage 1 | Stage 2 | Stage 3 | Stage 4 | Stage 5 |
| Alignment | UR1, UR2 | UR1, | UR1, | UR1, | green | green |
| | red | UR2 | UR2 | UR2 | | |
| | | red | yellow | yellow | | |
| Edge | UR1 | UR1 | UR1 | UR1 | green | green |
| alignment | red | red | red | yellow | | |
| Buccolingual | LL4, LR5 | LL4, | LL4, | LL4, | green | green |
| inclination | red | LR5 | LR5 | LR5 | | |
| | | yellow | yellow | yellow | | |
| Class | green | green | green | green | green | green |
| relationship | | | | | | |
| Leveling | green | green | green | green | green | green |
| Mesial/distal | green | green | green | green | green | green |
| tilt | | | | | | |
| Relative | green | green | green | green | green | green |
| midline | | | | | | |
| Absolute | red | red | red | yellow | green | green |
| midline | | | | | | |
| Occlusal | LL6, UL6 | LL6, | LL6, | LL6, | LL6, | green |
| contacts | red | UL6 | UL6 | UL6 | UL6 | |
| | | red | red | yellow | yellow | |
| Overbite | yellow | yellow | yellow | yellow | yellow | green |
| Overjet | green | green | green | green | green | green |
| Canine | green | green | green | green | green | green |
| overbite | | | | | | |
| Spacing | green | green | green | green | green | green |
| Collision | green | green | green | green | green | green |
| Contact | green | green | green | green | green | green |
| Bite | green | green | green | green | green | green |
| symmetry | | | | | | |
| Arch form | green | green | green | green | green | green |
| shape | | | | | | |
| Arch form | green | green | green | green | green | green |
| symmetry | | | | | | |

### Interactive Heatmap

Heat maps could be designed with active fields: Users could click on a field and get a preview of the implicated teeth as shown in the example below.

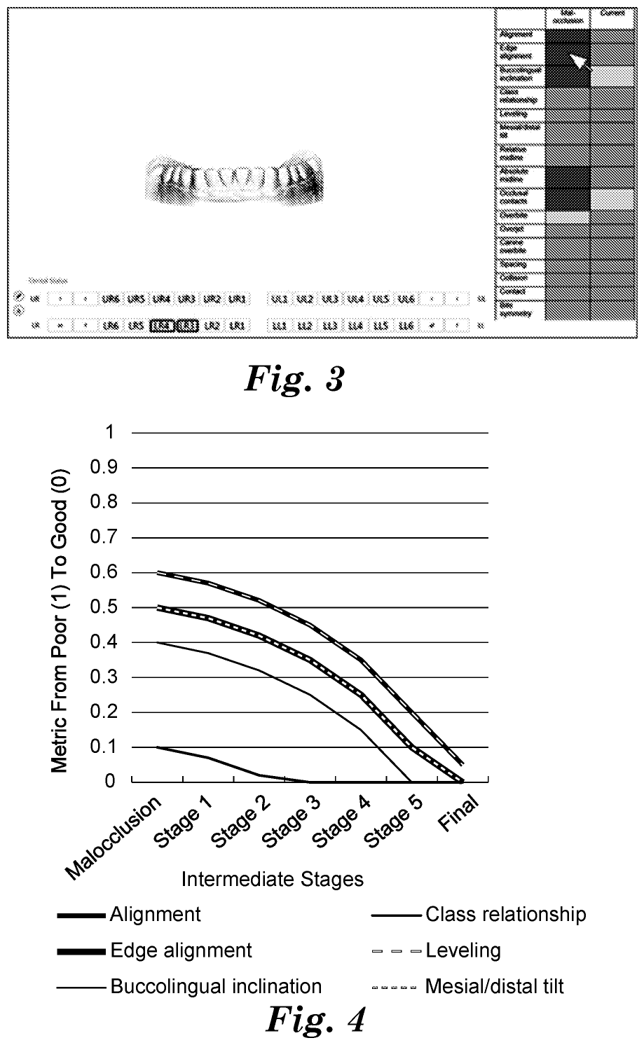

Example: User selects edge alignment field on heat map at the right side of the dashboard and implicated teeth UR4 and UR3 are highlighted in the tooth overview at the bottom of the dashboard. FIG. 3 is an example of a user interface displaying a heat map for orthodontic metrics.

### Trends

Trend data is informative for tracking treatment modifications. Improvements can be indicated by upward arrows, and worsening with downward arrows. This could be integrated with a heatmap.

Example: Display treatment planning over time/stages to patient with tooth and trend information: Shown is an example where one tooth (UR1) is initially moved out of a good alignment position to make room for another tooth (UR2, before both teeth are moved into a good alignment position

| | Mal- | | | | | |
|---|---|---|---|---|---|---|
| | occlusion | Stage 1 | Stage 2 | Stage 3 | Stage 4 | Stage 5 |
| Alignment | UR1 [symbol] | UR1 [symbol] | URA [symbol] | URl [symbol] | green | green |
| | UR2 | UR2 | UR2 | UR2 | | |
| | red | red | yellow | yellow | | |

### Line Graphs

Line graphs can be used to display treatment development over time. For instance, initial movements may be slower than later ones, as displayed in the example. FIG. 4 is an example of a user interface displaying a line graph showing treatment development at each intermediate stage.

### Bar Graphs

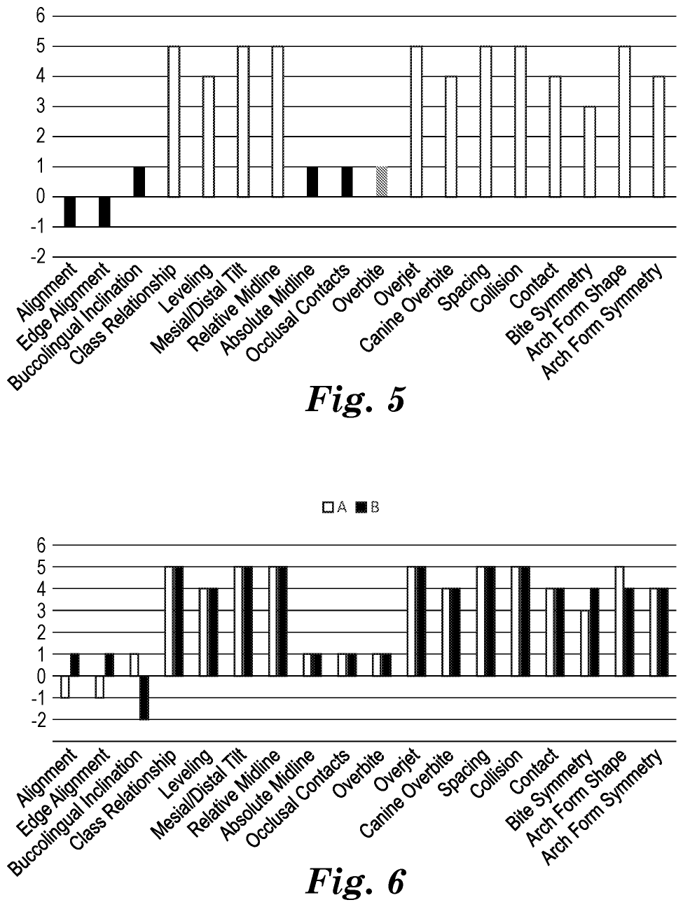

Technicians or doctors may desire a more complex display than a heatmap. Bar graphs could be used for this purpose. FIG. 5 is an example of a user interface displaying a bar graph tracking current setup, and FIG. 6 is an example of a user interface displaying a bar graph comparing two treatment plans.

Arch with Heat Map

Metrics can also be visualized to a clinician or patient in an interactive tool by generating a heat map on the arch. For example, bite comfort is a display of the collisions and penetration depths at all locations along the arch. Large penetration depths indicate a poorly aligned bite and may be an early indicator of occlusal disease. Thus, this tool can help a patient and clinician choose among a set of orthodontic setups to identify a setup that minimizes risk of future damage. This tool could also be used to help a dentist plan occlusal treatment (e.g., addition or removal of material) or implants by displaying a tooth arch heat map to show how bite comfort would be affected following treatment.

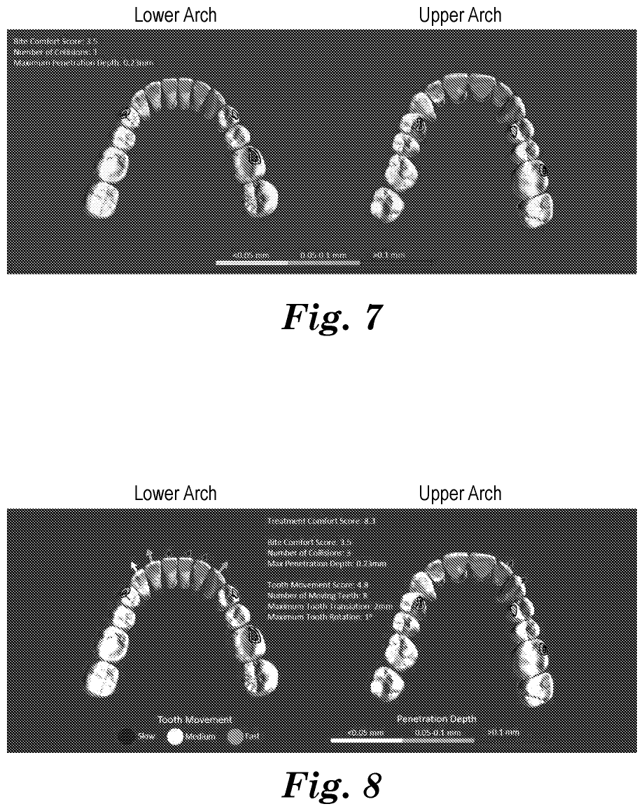

FIG. 7 is an example of a user interface displaying information about bite comfort based upon between-arch collisions, and FIG. 8 is an example of a user interface displaying information about bite comfort based upon between-arch collisions and tooth movement speed. Bite alignment metrics, as well as distances between landmark points, could be included in the display shown in FIG. 8.

### Analyzer: Metrics for Visualization

To achieve a simplified display, for example, underlying metrics need to specified.

### Metrics Thresholds: Manual

The acceptable thresholds for each metric could be specified manually by a user or prescribed by a doctor.

### Metrics Thresholds: Standard Values

Standard values for each metric could be learned from past patient final setups, and measurements that deviate from the standard values by a large amount (e.g., midline discrepancy larger than 95% of the population) would be highlighted to be revisited. Deviation from a standard value could be computed as:

(Measurement−Mean)/Standard Deviation (Eq. 1)

or, using a non-parametric approach:

(Measurement−Median)/Inner-Quartile Range (Eq. 2)

or:

Count(Population Metric>Measurement)/Count(Population) (Eq. 3)

Equation 3 describes the percentile score of the setup relative to the population (e.g., what percent of the population has a score that is better than the score of the current setup). The percentile score could change color based on how close to “normal” the current score is. For instance, green if the case is better than >90% of the population, yellow if it is better than >50% of the population, red otherwise.

1. A computer-implemented method for evaluating setups for orthodontic treatment, comprising steps of:
receiving one or more setups, each setup being a digital representation of a state of teeth at a particular stage of orthodontic treatment;
computing one or more scores based upon one or more metrics related to at least some of the states represented by the corresponding setups, wherein the scores provide an indication of a validity of the corresponding states; and
displaying the one or more metrics, or an indication of the corresponding one or more scores, or both the one or more metrics and the indication of the one or more scores.

2. The method of claim 1, wherein the displaying step comprises displaying a status of modifications of the orthodontic treatment.

3. The method of claim 2, wherein the displaying step comprises displaying different colors to indicate the status.

4. The method of claim 1, wherein the displaying step comprises displaying a status of a comparison of different options for the orthodontic treatment.

5. The method of claim 4, wherein the displaying step comprises displaying different colors to indicate the status.

6. The method of claim 1, wherein the displaying step comprises displaying a status of intermediate stages of the orthodontic treatment or the one or more final setups.

7. The method of claim 6, wherein the displaying step comprises displaying different colors to indicate the status.

8. The method of claim 6, wherein the displaying step comprises displaying tooth information for one or more of the stages.

9. The method of claim 6, wherein the displaying step comprises displaying trends for one or more of the stages.

10. The method of claim 1, wherein the displaying step comprises displaying a line graph of development of the orthodontic treatment over time.

11. The method of claim 1, wherein the displaying step comprises displaying a bar graph of development of the orthodontic treatment over time.

12. The method of claim 1, wherein the displaying step comprises displaying a bar graph of a comparison of different options for the orthodontic treatment.

13. The method of claim 1, wherein the displaying step comprises displaying a heat map providing a visual indication of one or more of the metrics.

14. The method of claim 1, further comprising receiving a digital representation of a malocclusion corresponding with the one or more intermediate and final setups.

15. The method of claim 1, further comprising providing auditory information corresponding with the one or more metrics, or the indication of the corresponding one or more scores, or both the one or more metrics and the indication of the one or more scores.

16. The method of claim 1, wherein the receiving step comprises receiving one or more intermediate setups.

17. The method of claim 1, wherein the receiving step comprises receiving one or more final setups.

18. The method of claim 1, wherein the receiving step comprises receiving one or more intermediate and final setups.

19-21. (canceled)
